(19) United States
(12) Patent Application Publication
(10) Pub. No.: US 2026/0272704 A1
(43) Pub. Date: Sep. 17, 2026

(54) Cooling Devices and Related Methods

(71) Applicant: Neuraxis, LLC, Sandown, NH (US)

(72) Inventors: Michael Drnek, Hampstead, NH (US); Dan Farley, Traverse City, MI (US); Callan Kennedy, Portsmouth, NH (US); Hailey McDonnell, Methuen, MA (US); Mia Banglmaier, Dover, NH (US)

(21) Appl. No.: 19/563,718

(22) Filed: Mar. 11, 2026

Related U.S. Application Data
(60) Provisional application No. 63/770,121, filed on Mar. 11, 2025.

(51) Int. Cl. A61F 7/00; A61B 17/02; A61B 17/00

(52) U.S. Cl. CPC A61F 7/00; A61B 17/02; A61B 2017/00477; A61F 2007/0056

(57) ABSTRACT

Cooling device(s), systems and related methods are disclosed. The cooling device(s) and systems may be configured to operate as a unit of a larger cooling system. The cooling device(s) and systems may have one or more inlets and outlets and one or more lumens extending within the cooling device(s). The combination of inlets, outlets, and lumens may form a fluid pathway within the cooling device(s). The cooling device(s) may include one or more additional features for securing or interfacing with a patient before, during, and/or after a surgical procedure. The method(s) may placing or otherwise coupling the cooling device(s) with a patient and circulating cooling fluid from a cooling system into the cooling device(s) to apply thermal therapy to an area of interest of a patient.

### CROSS-REFERENCE TO RELATED APPLICATIONS

This patent application is a Utility Application claiming benefit of U.S. Provisional Patent Application No. 63/770,121, titled “Cooling Devices and Related Methods,” filed on Mar. 11, 2025, which is herein incorporated by reference in its entirety.

### BACKGROUND

Cooling devices present numerous benefits in the field of patient care. For example, the application of certain degrees of hypothermia to an area of a patient’s body during or after injury can lead to benefits such as a reduction of the metabolic demand of various cells, reduction of edema, added tolerance to hypoxia/ischemia, and ultimately a reduction in damage to the patient’s body. The use of a cooling effect for these purposes can be referred to as therapeutic hypothermia.

Retractor blades are used during surgery to hold incisions opening by retracting soft tissue and allowing the surgeon to work within the surgical site. Various embodiments of retractor blades exist, including hand-held, self-retaining, and table mounted retractor blades. Retractor blade systems are commonly used for spine procedures. However, retractor blades are known for causing added injury in use during procedures. For example, known retractor blade associated complications include inflammation, nerve damage, and pain. Specifically, retraction of tissue during spinal surgeries commonly causes pain post-surgery.

### SUMMARY

The subject matter of this application may involve, in some cases, interrelated products, alternative solutions to a particular problem, and/or a plurality of different uses of a single system or article.

In accordance with some embodiments a cooling system for applying thermal therapy to a tissue of a patient is disclosed. The cooling system may include a retractor blade. The retractor blade may include an interfacing location extending along at least a portion of a length of the retractor blade and a cooling attachment configured to couple with the interfacing location. The attachment may include an inlet configured to interface and be in fluid communication with a fluid supply; a lumen in fluid communication with the inlet and extending at least partially within the cooling attachment; and an outlet in fluid communication with the lumen and configured to interface and be in fluid communication with a fluid return.

In some additional embodiments, the cooling system the interfacing location is on a tissue contacting side of the retractor blade.

In some additional embodiments, the cooling attachment is engaged with the interfacing location by an interfacing technique selected from a group consisting of: snap-fit, slot-fit, adhesives, fasteners, or combinations thereof.

In some additional embodiments, the cooling attachment is snap-fit within the interfacing location.

In some additional embodiments, the cooling attachment further includes an insert configured to couple with the blade; and carry fluid within at least one lumen disposed within the insert. The terminal cooling element is configured to interface and be in fluid communication with an insert; and couple to the interfacing location.

In some additional embodiments, the terminal cooling element comprises a cavity configured to receive the insert and enable fluid communication between the lumen of the insert and a lumen of the terminal cooling element.

In some additional embodiments, the terminal cooling element has a larger volume of lumen than the insert.

In some additional embodiments, the terminal cooling element comprises metal.

In some additional embodiments, the terminal cooling element comprises a fluid reservoir distal from an interfacing location of the insert.

In accordance with some embodiments a cooling device for applying thermal therapy to a tissue of a patient is disclosed. The cooling device may include a body; an inlet disposed on the body and configured to interface and be in fluid communication with a fluid supply; a lumen in fluid communication with the inlet and extending at least partially within the body; an outlet disposed on the body and in fluid communication with the lumen and configured to interface and be in fluid communication with a fluid return; and at least one holding element disposed on the body and configured to retain the cooling device in a position when the cooling device is interfaced with the patient.

### BRIEF DESCRIPTION OF THE DRAWINGS

FIGS. 1A(i) – FIG. 1A(v) illustrate an exemplary patient cooling device coupled with a blade. FIG. 1A(i) is a side view of the patient cooling device coupled with a blade. FIG. 1A(ii) shows a back view of the blade coupled with the patient cooling device and with a tube slot configured to aid in coupling the patient cooling device with the blade. FIG. 1A(iii) is a perspective view of the patient cooling device coupled with the blade showing a curved profile of the patient cooling device which is flush with the profile of the blade. FIG. 1A(iv) is a back view of the patient cooling device coupled with the blade showing a slot configured to permit coupling of the cooling device with the blade. FIG. 1A(v) is a perspective front view of the patient cooling device coupled with the blade.

FIGS. 1B(i) –FIG. 1B (v) illustrate a patient cooling device of FIGS. 1A(i) – FIG. 1A(v) configured to couple with a retractor blade. FIG. 1B(i) is a side view of the patient cooling device. FIG. 1B(ii) is a front of the patient cooling device showing a docking mechanism disposed on an end of the patient cooling device. FIG. 1B(iii) is a perspective view of the patient cooling device. FIG. 1B(iv) is a back perspective view of the patient cooling device showing a concave shape of the patient cooling device. FIG. 1B(v) is a perspective view of the patient cooling device.

FIGS. 2A(i) – FIG. 2A(v) illustrate another exemplary patient cooling device. FIG. 2A(i) is a side view of the patient cooling device. FIG. 2A(ii) is a perspective side view of the patient cooling device. FIG. 2A(iii) is a front view of the patient cooling device. FIG. 2A(iv) is a perspective back view of the patient cooling device. FIG. 2A(v) is a back view of the patient cooling device.

FIGS. 2B(i) – FIG. 2B(v) illustrate the patient cooling device of FIGS. 2A(i) – 2A(v) coupled with a blade. FIG. 2B(i) is a side view of the patient cooling device coupled with a blade. FIG. 2B(ii) is a perspective front view of the blade with the patient cooling device coupled with the blade. FIG. 2B(iii) is a front view of the blade with a coupled patient cooling device showing no tube slot on the blade. FIG. 2B(iv) is a back perspective view of the blade with a coupled patient cooling device. FIG. 2B(v) is a back view of the blade with a coupled patient cooling device.

FIGS. 3A(i) –FIG. 3A (v) illustrate another exemplary patient cooling device coupled with a blade. FIG. 3A(i) is a side view of the patient cooling device coupled with the blade. FIG. 3A(ii) is a front view of the blade with a coupled patient cooling device. FIG. 3A(iii)is a front perspective view of the blade with a coupled patient cooling device. FIG. 3A(iv) is a back view of the blade with a coupled patient cooling device. FIG. 3A(v) is a back perspective view of the blade with a coupled patient cooling device.

FIGS. 3B(i) – FIG. 3B(vii) illustrate the blade without the patient cooling device of FIGS. 3A(i) – 3A(v) coupled to the blade. FIG. 3B(i) is a side view of the blade. FIG. 3B(ii) is a front view of the blade configured to accept a patient cooling device. FIG. 3B(iii) is a perspective front view of the blade showing the back face of the blade cutout to accept the cooling device. FIG. 3B(iv) is another perspective front view of the blade. FIG. 3B(v) is a back view of the blade. FIG. 3B(vi)is a perspective back view of the blade. FIG. 3B(vii) is a top view of the flange of the blade showing a location for tube coupling of the patient cooling device with the blade.

FIGS. 4A(i) – FIG. 4A(v) illustrate another exemplary patient cooling device coupled with a blade having no flange. FIG. 4A(i) is a side view of the patient cooling device coupled with the blade. FIG. 4A(ii) is a front view of the blade coupled to the patient cooling device. FIG. 4A(iii) is a perspective front view of the blade coupled to the patient cooling device coupled. FIG. 4A(iv) is a back view of the blade coupled with the patient cooling device. FIG. 4A(v) is a perspective back view of the blade coupled with the patient cooling device.

FIGS. 5A(i) – FIG. 5A(iv) illustrate another exemplary patient cooling device. FIG. 5A(i) shows a patient cooling device having an elongated body and comprising one or more appendages. FIGS. 5A(ii) – 5A(iii) show a circled and enlarged portion of the cooling device having the appendages. FIG. 5A(iv) is a side perspective view of the patient cooling device.

FIGS. 5B(i) – FIG. 5B(iv) illustrate another exemplary patient cooling device. FIG. 5B(i) shows the patient cooling device having an elongated body and comprising one or more appendages. FIGS. 5B(ii) – 5B(iii) show a circled and enlarged portion of the cooling device having the appendages. FIG. 5B(iv) is a side perspective view of the patient cooling device.

FIGS. 5C(i) –FIG. 5C(vi) illustrate another exemplary patient cooling device. FIG. 5C(i) shows the patient cooling device having an elongated body and comprising one or more appendages. Moreover, the circled portion of FIG. 5C(i) is the enlarged view of FIG. 5C(iv). FIG. 5C(ii) is a side view of the patient cooling device. FIG. 5C(iii) is a side perspective view of the patient cooling device. FIG. 5C(v) is a top perspective view of the patient cooling device. FIG. 5C(vi) is another side perspective view of the patient cooling device.

FIG. 6 is a diagram of a method of using the patient cooling device coupled with a blade during a surgical procedure.

FIG. 7 is a diagram of a method of using the patient cooling device of FIGS. 5A(i) – 5C(vi) during a surgical procedure.

It should be appreciated that features illustrated in the accompanying figures may not necessarily be drawn to scale and that the geometry depicted in the figures can vary from the images shown. Alternative arrangements and adjustments of components shown may be within the reach of one skilled in the art and the figures of the subject application are not meant to limit the disclosure to the particular embodiments depicted.

### DETAILED DESCRIPTION

### General Overview

In accordance with some embodiments of the present disclosure, cooling devices, cooling systems, and related techniques and methods are disclosed. The cooling devices/systems may be configured for use in surgery, and more specifically, for targeted cooling of anatomical features of a patient. Numerous types of devices capable of receiving a cooling fluid are described herein, such as, but not limited to, tissue retractor blades, malleable devices, and other devices. To such ends, the cooling devices/systems may include or be configured to interface with an inlet and an outlet. The inlet and the outlet may be configured to be in fluid communication with a fluid source and allow circulation of the cooling fluid within the cooling devices. In this way, the cooling device may provide thermal therapy to a desired location within a patient. In other embodiments, the tissue retractor blades, malleable devices, sheaths, and other devices may be configured or designed to interface with a cooling device, an insert, and/ or cooling element which may comprise the inlet, outlet and lumens. In such circumstances, the insert and/or cooling device may be designed to interface with the cooling fluid source and circulate the cooling fluid within lumens in order to cool a cooling device interfaced with another tool/system.

Generally, the disclosed cooling devices/systems may be configured for use with any of a wide range of surgical procedures. In accordance with some embodiments, the disclosed cooling devices/systems may be configured to be at least partially inserted into an incision, opening, and/or cavity of a patient, or otherwise be configured to interface and/or abut a portion of the body of the patient, whether interior or exterior. In accordance with some embodiments, the disclosed cooling devices/systems, and/or elements of the cooling devices/systems, may be configured for single use only, and, generally, may be disposable. In still some other embodiments, the disclosed cooling devices/systems and/or the elements of the cooling devices/systems may be configured for more than one use, and, generally, may be configured to facilitate proper sanitation processes of the cooling devices. In still some additional embodiments, the disclosed cooling devices may be configured to attach to any number of tissue retractor blades, malleable devices, sheaths, and/or drainage tubes, thereby creating a cooling system. Moreover, the disclosed device(s) are configured to facilitate clinical use and ensure sterility.

Some embodiments may realize one or more advantages or benefits, as compared to existing approaches. For example, in accordance with some embodiments, the disclosed cooling devices may provide cooling to tissue locations of a patient which, historically, lacked cooling during surgical procedures. Some embodiments disclosed herein may, in part or in whole, borrow from other embodiments as one skilled in the art would understand.

### Cooled Blade System

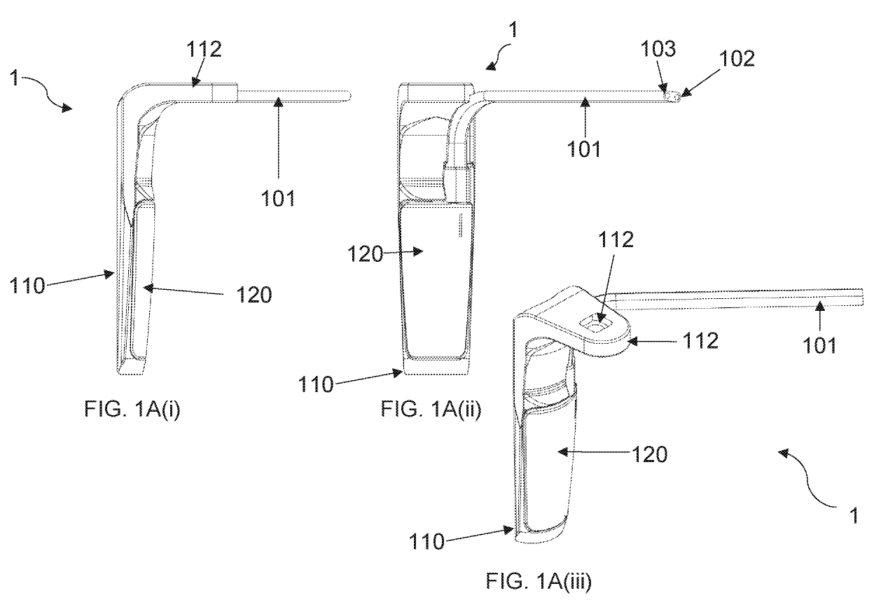

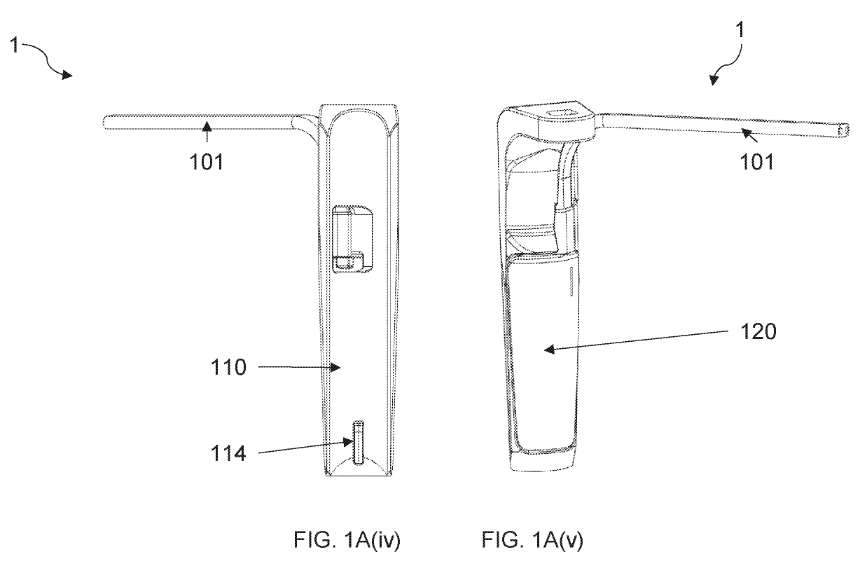

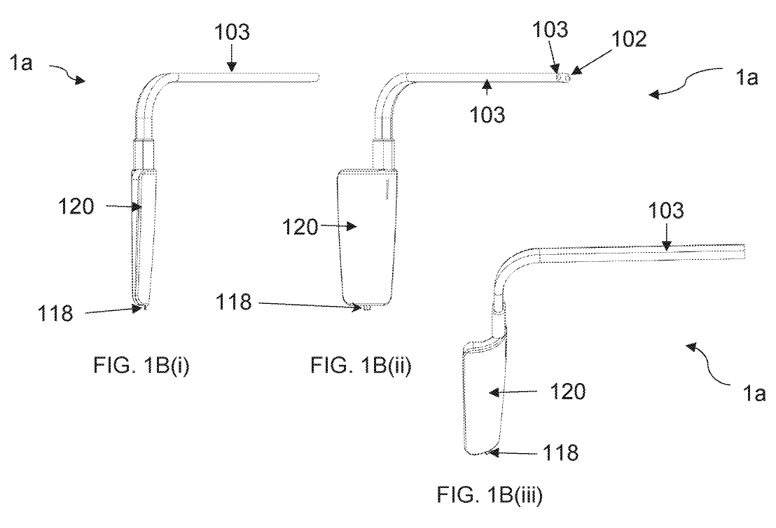

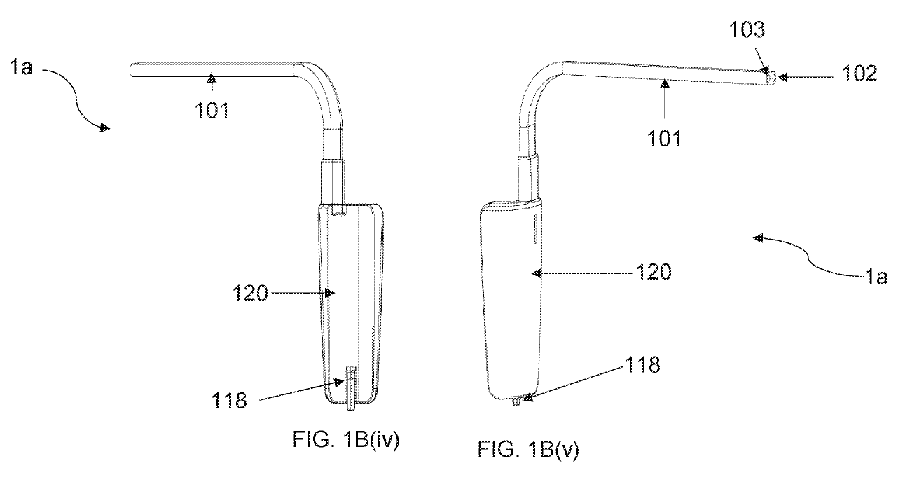

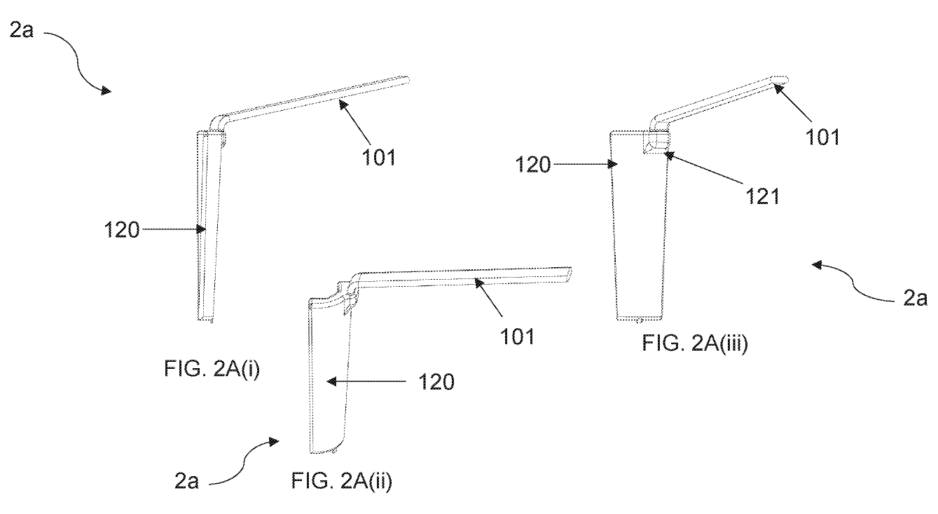

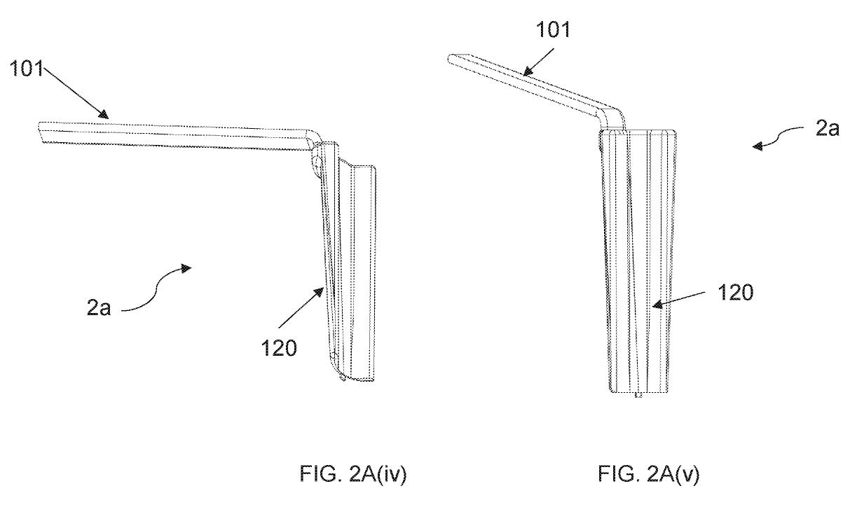

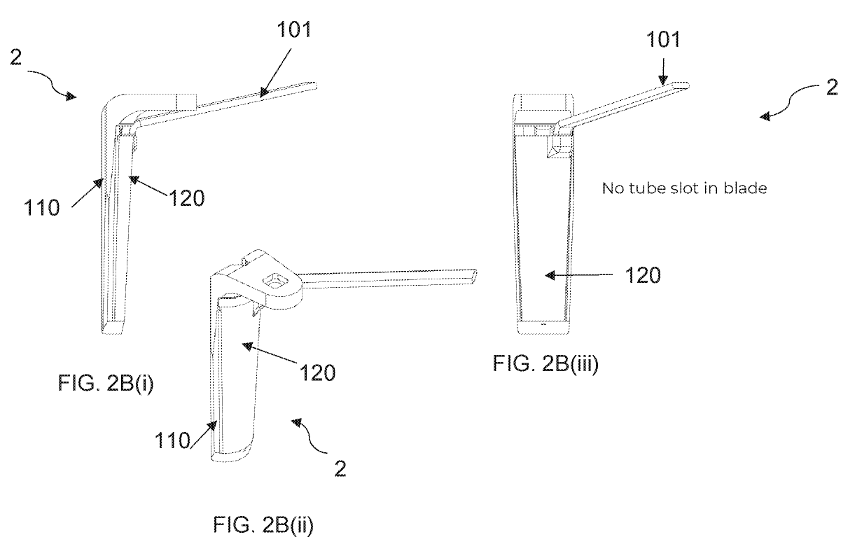

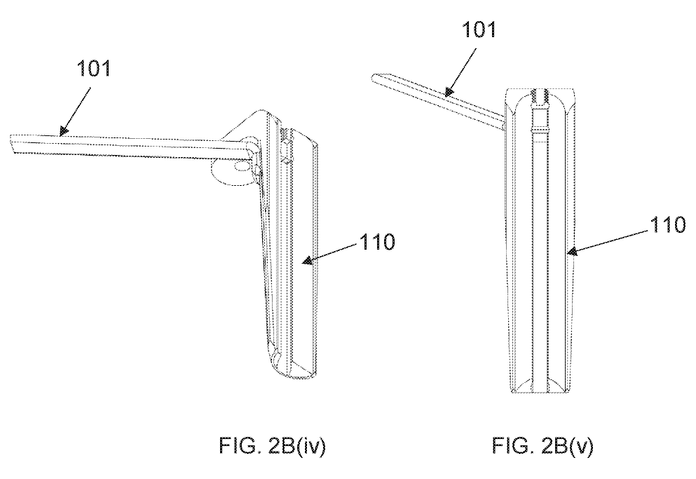

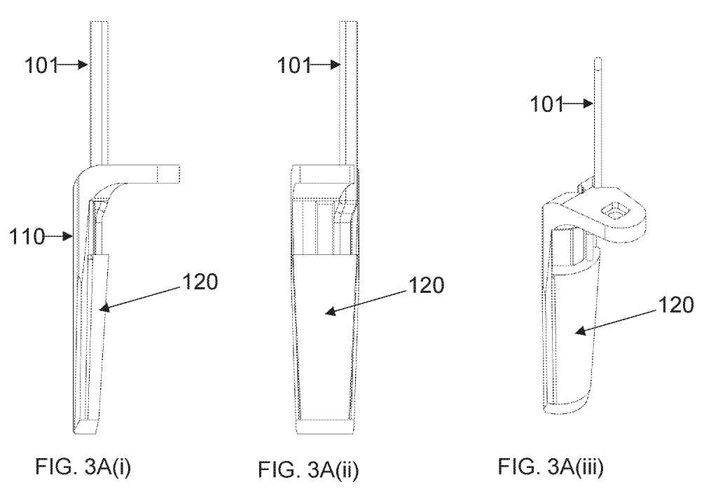

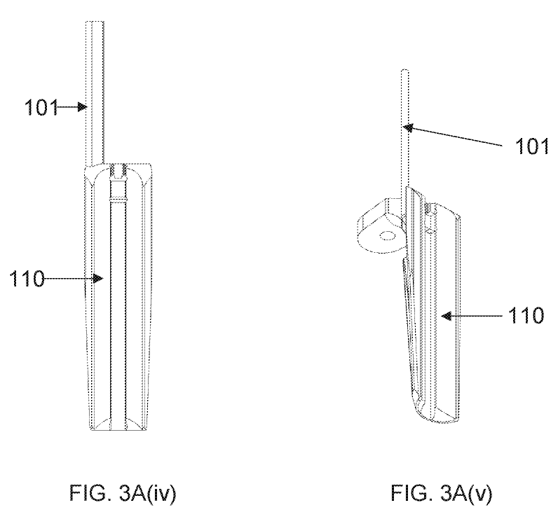

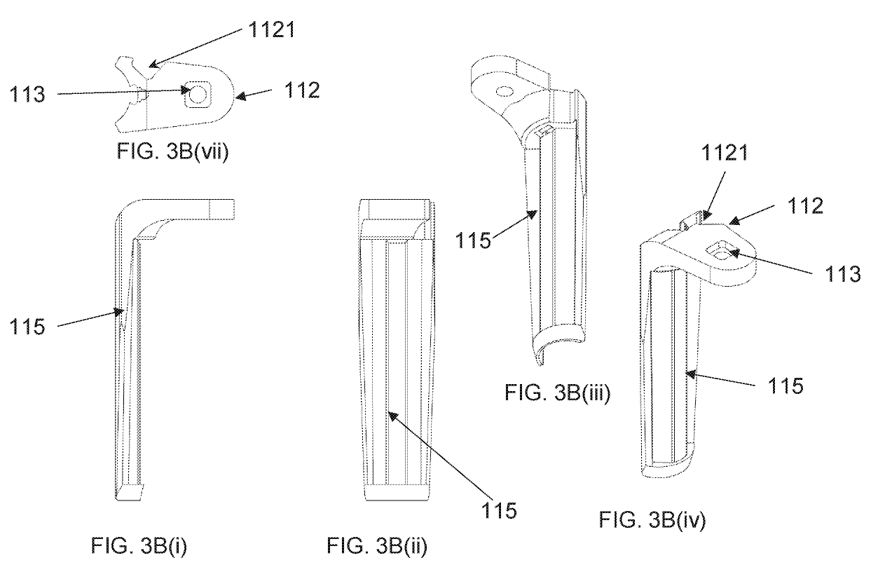

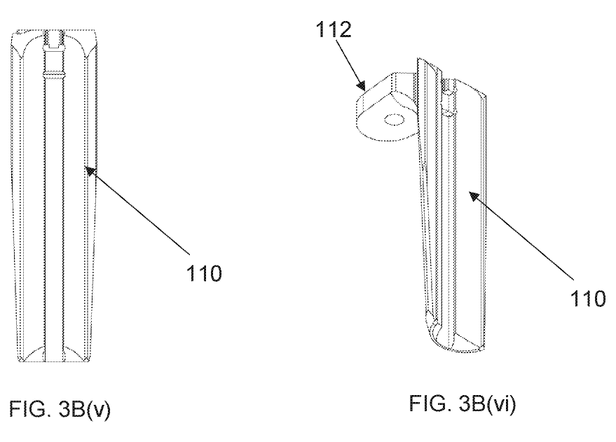

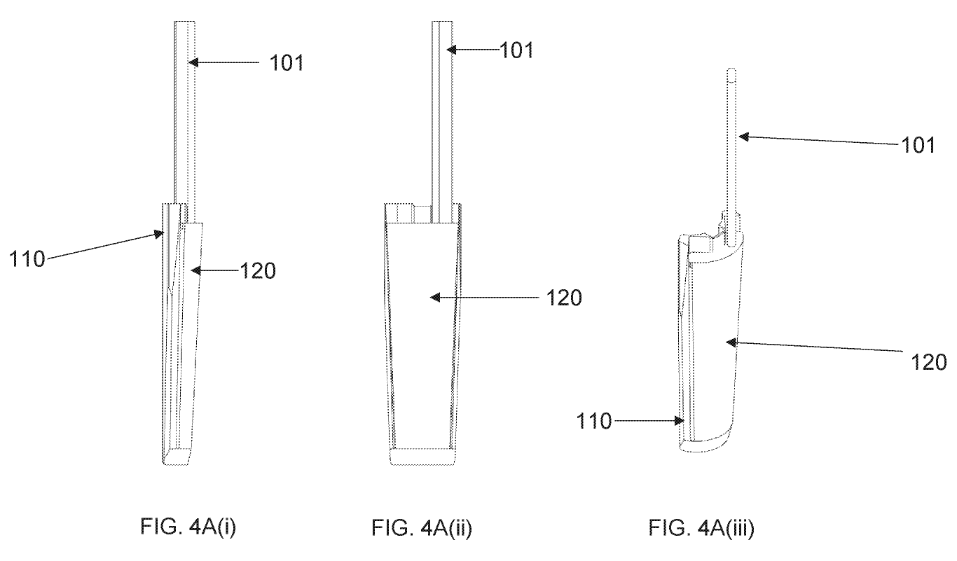

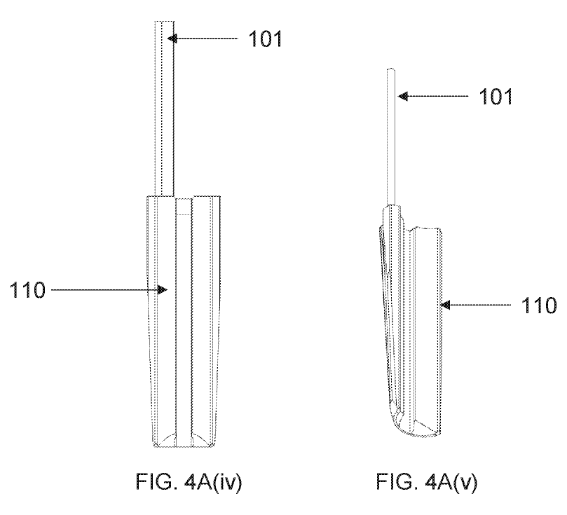

FIGS. 1A(i) – 4A(v) show various exemplary embodiments of cooling system(s) disclosed herein. More specifically, FIGS. 1A(i) – 1A(v) show an exemplary embodiment of cooling system 1, FIGS. 2B(i) – 2B(v) show an exemplary embodiment of cooling system 2, FIGS. 3A(i) – 3A(v) show an exemplary embodiment of cooling system 3, and FIGS. 4A(i) – 4A(v) show an exemplary embodiment of cooling system 4. It is to be understood that some features discussed with respect to some embodiments of cooling systems 1, 2, 3, and/or 4 may be common to other embodiments systems 1, 2, 3, and/or 4. Accordingly, common reference numerals and/or descriptions are used herein to describe the various features of cooling systems 1, 2, 3, and/or 4. Generally, for embodiments within cooling systems 1, 2, 3, and/or 4, the patient cooling device 1a, 2a, 3a, and/or 4a, may refer to the combination of insert 101 and terminal cooling element 120 and the cooling systems 1, 2, 3, and/or 4 may generally refer to when the patient cooling device 1a, 2a, 3a, and/or 4a is interfaced, coupled, attached, or otherwise joined with blade 110.

In accordance with some embodiments, the cooling systems 1, 2, 3, and/or 4 may include blade 110, insert 101, and/or terminal cooling element 120. Blade 110 may be configured to interface with insert 101 and/or terminal cooling element 120. In some additional embodiments, insert 101 may be integral with or be configured to couple with one or more terminal cooling element 120. In some embodiments, insert 101 may include inlet 102 and outlet 103. Inlet 102 and outlet 103 may be configured to be in fluid communication with a fluid source (not shown), a fluid dump, fluid pump, and/or a fluid recirculator. In further embodiments, insert 101 may include one or more lumens interfaced with, or otherwise in fluid communication with, inlet 102 and/or outlet 103. In accordance with some embodiments, the one or more lumens may extend within insert 101 and form a continuous fluid pathway from inlet 102 to outlet 103 within insert 101. Moreover, the one or more lumens may be in fluid communication with one or more lumens within terminal cooling element 120. Each of these elements is discussed in depth below.

In accordance with some embodiments, the cooling systems 1, 2, 3, and/or 4 may include blade 110. In accordance with some embodiments, blade 110 may be manufactured from one or more of the following materials: stainless steel, aluminum, titanium, polymer composites, or biocompatible alloys, or combinations thereof. In some cases, blade 110 may be substantially rigid. In accordance with some other cases, blade 110 may be substantially flexible. In still some more embodiments, blade 110 may comprise malleable sections, rigid sections, or a combination thereof to allow for both flexibility and structural stability.

Also, the longitudinal extent (e.g., length) and transverse extent (e.g., width or diameter) of blade 110 may be customized, as desired. In some embodiments, blade 110 may have a length in the range of about 20mm – 300mm (e.g., about 20 – 50 mm, about 50 – 150 mm, about 150 – 300mm, or any other subrange in range of about 20 – 300 mm). In some embodiments, blade 110 may have a width or diameter in the range of about 10 – 40 mm (e.g., about 10 – 20 mm, about 20 – 30 mm, about 30 – 40mm, or any other subrange in range of about 10 – 40 mm). In some embodiments, blade 110 may have a depth or thickness in the range of about 2 – 6 mm (e.g., about 2 – 3 mm, about 3 – 4 mm, about 4 – 6mm, or any other subrange in range of about 2-6 mm). In some embodiments, blade 110 may have a substantially uniform width, diameter, and/or depth along its entire length. In some additional embodiments, blade 110 may have a non-uniform (e.g., tapered, flared, ribbed, notched, constricted, bulged, or otherwise varying) width, diameter, and/or depth along its entire length. As will be appreciated in light of this disclosure, it generally may be desirable to ensure that blade 110 is sufficiently sized and of appropriate geometry to permit interfacing with insert 101, terminal cooling element 120, and/or a patient body/cavity.

Also, as can be seen from FIGS. 1Ai) – 1A(v), in some embodiments, blade 110 may be of generally rectangular shape. In some cases, as can seen from FIGS. 3B(i) – 3B(vi), blade 101 may include a beveled and/or tapered section. In some cases, the beveled and/or tapered section may decrease the transverse extent of blade 110 along at least a portion of the blade’s length. In some additional cases, the beveled and/or tapered section may decrease the thickness of the blade along at least a portion of the blade’s length and/or width. In some more cases, the beveled and/or tapered section may decrease both the transverse extent and the thickness of the blade along at least a portion of the blade’s length. In some instances, the beveled and/or tapered section may extend along a partial or full extent of blade 101’s width.

In some additional cases, blade 110 may include a flange 112. In some cases, flange 112 may extend at angle in the range of about 30 – 120 degrees from the blade 110. Generally, the flange 112 may be sufficiently sized and of appropriate geometry to allow fixation of the blade 110 to one or more mounting systems (not shown). In some additional cases, flange 112 may extend substantially perpendicular from blade 110. In some embodiments, flange 112 may be configured to facilitate removal of an insert 101 (explained below) and/or interface with a mounting system (not shown) to mount and/or hold blade 110. In some cases, the mounting system may be a table mounting system or another system used in close proximity to the surgery site to allow a user to securely position the blade 110 while blade 110 is in use. In some additional cases, the mounting system may be configured to mount and/hold the blade 110 at a different location other than the flange 112.

In some embodiments, as can be seen in FIG. 1A(iii), the flange 112 may include one or more hole(s) 113. In other embodiments, the flange 112 may not include the one or more hole(s) 113. In some embodiments, the one or more hole(s) may be generally polygonal (e.g., triangular, rectangular, square, etc.) in cross-sectional shape. In some additional embodiments, the one or more hole(s) may generally be circular, elliptical, semi-circular, or semi-elliptical in cross-sectional shape. In some cases, the one or more hole(s) may have a uniform shape or have a combination of cross-sectional shapes, in whole or in part. For example, in some instances the one or more hole(s) may have a uniform diameter throughout the extent of the one or more hole(s). In some other instances, the diameter of the one or more hole(s) may change (e.g., increase or decrease) along the depth of the one or more hole(s). Other suitable types, or combination of types, of cross-sectional shapes, as described herein, will depend on a given target application or end-use and will be apparent in light of this disclosure. In accordance with some embodiments, the one or more holes may be configured to permit a device or tool to be inserted therethrough to release insert 101 from track 115 (discussed below).

In some embodiments, as can be seen in FIG. 3B(vii), the flange 112 may include an insert snap element portion 1121 formed at one side on the flange 112. The insert snap element portion 1121 may be configured to permit interfacing, coupling, snapping, or otherwise attaching at least a portion of insert 101 to flange 112, thereby making an extra securing option for insert 101 on flange 112. Generally, the insert snap element portion 1121 may be defined by one or more side walls of flange 112, thereby creating an empty space for insert 101 to be maneuvered into.

In accordance with some embodiments, as can be seen at least in FIGS. 1A(ii) and 1A(v), the blade 110 may include an insert slot 116, configured to allow coupling between blade 110 and insert 101. For instance, in some cases, insert slot 116 may allow at least a portion of insert 101 to be inserted into blade 110. The insert slot 114 may be formed on the same side of blade 110 as where insert 101 and terminal cooling element 120 may be configured to couple with blade 110. In some other embodiments, as can be seen in FIGS. 2B(i) – 2B(v), blade 110 may not include an insert slot 116, and instead insert 101 may be configured to interface only to terminal cooling element 120 and one or more external cooling systems for delivering the fluid to the insert 101.

In accordance with some embodiments, and as can be seen from FIGS. 3B(ii) – 3B(iv), blade 110 may include track 115 extending along at least a part of a length of blade 110. In some embodiments, track 115 may be configured to interface with one or more insert(s) 101 and/or one or more terminal cooling elements 120 (as described below). In some cases, track 115 may be configured to be on a tissue-contacting side, or non-operating side, of blade 110. In other cases, track 115 may be configured to be on the non-tissue contacting side, or the operating side, of blade 110. In accordance with some embodiments, when insert 101 is be interfaced with blade 110, the profile of insert 101 may not be within the working path or does not obstruct the view of a surgical site. In still more embodiments, insert 101 may be configured to be flush with or protrude from a side and/or a face of the blade 110. Generally, the track 115 is configured as a mating face with appropriate geometries to permit, ensure, allow, or otherwise provide an interfacing location for one or more inserts 101 and/or terminal cooling element 120. In some additional cases, the track 115 may be disposed on the blade 110 such that the track is at least partially sunken into the blade 110. In some more cases, the track 115 may be an interfacing location between two walls extending at least partially from the face of the blade 110, such that the insert(s) 101 and/or terminal cooling element 120 may abut the face of the blade 110 when interfaced with the track.

Also, the longitudinal extent (e.g., length) and transverse extent (e.g., width or diameter) of track 115 may be customized, as desired. In some embodiments, track 115 may have a length in the range about 20mm – 300mm (e.g., about 20 – 50 mm, about 50 – 150 mm, about 150 – 300mm, or any other subrange in range of about 20 – 300 mm). In some embodiments, track 115 may have a width or diameter in the range of about 10 – 40 mm (e.g., about 10 – 20 mm, about 20 – 30 mm, about 30 – 40mm, or any other subrange in range of about 10 – 40 mm). In some embodiments, track 115 may have a depth or thickness in the range of about 2 – 6 mm (e.g., about 2 – 3 mm, about 3 – 4 mm, about 4 – 6mm, or any other subrange in range of about 2-6 mm). For example, in some cases, track 115 may extend from a proximate end of blade 110 to a distal end of blade 110, in part or in whole. For example, in some cases, as seen in FIGS. 3B(i) – 3B(iv), track 115 may terminate before the end edge of blade 110, while, in some more cases, track 115 may continue through the end of blade 110. In some embodiments, track 115 may be shorter in width than blade 110 such that track 115 fits within the transverse extent of blade 110. In some additional embodiments, the track 115 may be substantially equal to the width of blade 110. In some embodiments, track 115 may have a substantially uniform width or diameter along its entire length. In some additional embodiments, track 115 may have a non-uniform (e.g., tapered, flared, ribbed, notched, constricted, bulged, or otherwise varying) width or diameter along all or a portion of its entire length. As will be appreciated in light of this disclosure, it generally may be desirable to ensure that track 115 is sufficiently sized and of appropriate geometry to permit insert 101 and/or terminal cooling element 120 to interface with blade 110.

In accordance with some embodiments, the profile of blade 110 may be concave, convex, or flat. In respective cases, track 115 may be configured to follow the profile of blade 110 and insert 101 and/or terminal cooling element 120 may be configured or otherwise able to interface with blade 110 regardless of blade 110’s profile. In some cases, track 115 may be biased towards one edge of blade 110, such that track 115 may be off-center on blade 110, or may be centered on blade 110.

In accordance with some embodiments, and as can be seen in FIG. 1A(iv), track 115 (and blade 110) may include one or more recess(es) 114. Generally, the size and shape of the recess(es) may be customized, as desired. In accordance with some embodiments, the recess(es) may be configured to attach, connect, or otherwise interface with a protrusion 118 of terminal cooling element 120 (described below).

In accordance with some embodiments, as can be seen from at least FIGS. 1A(i) – 3A(v), the cooling systems 1, 2, 3, and/or 4 may include insert 101. In accordance with some embodiments, and as can be seen from at least FIGS. 1A(i) – 1A(v), insert 101 may be configured to interface with blade 110. Generally, insert 101 may be, or may be configured as, tubing configured to transfer fluid through one or more lumens (discussed below) within insert 101. Therefore, generally, insert 101 may be a connection point bringing cooling fluid from an outside system to one or more terminal cooling elements 120 (discussed below). Accordingly, insert 101 may include one or more inlet(s) 102 and one or more outlet(s) 103 (as explained below). The inlet(s) 102 and outlet(s) 103 may be in fluid communication with one or more internal pathways (not shown) within the insert 101. The one or more of the internal fluid pathways may be referred to as lumens within insert 101.

As stated above, insert 101 may be configured to interface with blade 110. The interface between blade 110 and insert 101 may use one or more of the following securing methods: snap-fit, slide-fit, friction-fit, and slot-fit. In some additional embodiments, adhesive, screws, tape, or combination thereof may be used in conjunction with the securing methods to achieve a secure interface between blade 110 and insert 101. Other suitable types, or combination of types, of securing methods as described herein will depend on a given target application or end-use and will be apparent in light of this disclosure.

In accordance with some embodiments, insert 101 may be manufactured from one or more of the following materials: stainless steel, aluminum, titanium, polymer composites, or biocompatible alloys, or combinations thereof. In some cases, insert 101 may be substantially rigid. In accordance with some other cases, insert 101 may be substantially flexible. In still some more embodiments, insert 101 may comprise malleable sections, rigid sections, or a combination thereof to allow for both flexibility and structural stability. In some cases, insert 101 may be malleable when a force is applied to it, but may also be able to maintain a shape when force is not being applied.

Also, the longitudinal extent (e.g., length) and transverse extent (e.g., width or diameter) of insert 101 may be customized, as desired. In some embodiments, insert 101 may have a length in the range of about 20mm – 300mm (e.g., about 20 – 50 mm, about 50 – 150 mm, about 150 – 300mm, or any other subrange in range of about 20 – 300 mm). In some embodiments, insert 101 may have a maximum width or diameter in the range of about 10 – 40 mm (e.g., about 10 – 20 mm, about 20 – 30 mm, about 30 – 40mm, or any other subrange in range of about 10 – 40 mm). In some embodiments, insert 101 may have a substantially uniform width, diameter, and/or depth along its entire length. In some additional embodiments, insert 101 may have a non-uniform (e.g., tapered, flared, ribbed, notched, constricted, bulged, or otherwise varying) width, diameter, and/or depth along its entire length. As will be appreciated in light of this disclosure, it generally may be desirable to ensure that insert 101 is sufficiently sized and of appropriate geometry to permit interfacing with track 115 and/or blade 110.

As explained above, insert 101 may include one or more lumen(s). In accordance with some embodiments, the lumen(s) may be internal fluid pathways in fluid communication with one or more inlet(s) 102, one or more outlet(s) 102, and/or one or more additional lumen(s). Generally, the inlet(s) 102, the outlet(s) 102, and/or the one or more lumen(s) may be a part of a closed fluid loop with one or more additional cooling device(s), elements (such as terminal cooling element 120), and/or systems. In accordance with some embodiments, the lumen(s) may be embedded, in whole or in part, within insert 101. In some cases, the lumens may be circular, elliptical, star-shaped, oval, and/or rectangular in cross-sectional shape, in part or in whole. Other suitable types, or combinations of types, of cross-sectional shapes as described herein will depend on a given target application or end-use and will be apparent in light of this disclosure.

Also, generally the longitudinal extent (e.g., length) and transverse extent (e.g., width or diameter) of the lumens of insert 101 may be customized, as desired, and will depend on the overall dimensions of the insert 101. In some embodiments, the lumen(s) may have a substantially uniform width, diameter, and/or depth along its entire length. In some additional embodiments, the lumen(s) may have a non-uniform (e.g., tapered, flared, ribbed, notched, constricted, bulged, or otherwise varying) width, diameter, and/or depth along its entire length. In some additional embodiments, the lumen(s) may terminate in a reservoir located within the insert 101. As will be appreciated in light of this disclosure, it generally may be desirable to ensure that the lumen(s) are sufficiently sized and of appropriate geometry to permit fluid flow through the lumens within the insert 101.

In accordance with some embodiments, the shape of the lumen pathway, may be customized, as desired. In some embodiments, the lumen(s) may be generally straight, curved, zig-zagged, or otherwise formed to provide a pathway from one internal volume portion of the insert 101 to another internal volume portion of the insert 101. As will be appreciated in light of this disclosure, it generally may be desirable to ensure the one or more internal fluid pathway(s) (i.e., the one or more lumen(s)) are sufficiently designed to permit a transfer of thermal energy in an efficient manner.

In accordance with some embodiments, the one or more lumens may include a copper element extending along at least a partial extent of the one or more lumen(s), in whole or in part. The copper element may be positioned within the lumen, on an inside wall of the lumen, and/or on an outside wall of the lumen. The copper element may also be, in some cases, positioned adjacent to, proximate to, or distal from the lumen on and/or within insert 101. Generally, the copper element may be positioned anywhere on and/or within insert 101 to help aid in shaping and/or conforming insert 101 to a specific shape and/or retaining insert 101 in a shape. The copper element may be a thin strand of copper wire configured to help with contouring the insert 101 to the anatomy of the patient body part where the cooling device(s) is being used.

In accordance with some embodiments, the one or more lumen(s) may be in fluid communication at a distal end of insert 101 via a reservoir, a connecting tube, inlet(s) 102, outlet(s) 103, or combinations thereof. For example, in some cases, inlet 102 may be in fluid communication with a lumen which extends at least partially within insert 101. In some additional cases, the lumen(s) may be designed or configured to be in fluid communication with both inlet 102 and outlet 103 such that fluid may enter inlet 102, flow through the lumen(s) within insert 101 and then exit outlet 103. In some additional cases, the outlet(s) may be configured as inlet(s) and the inlet(s) may be configured as outlet(s). In yet another embodiment, the lumen(s) may be interfaced with a terminal cooling element 120, described below.

In accordance with some embodiments, as can be seen in at least FIGS. 1A(i) – 3A(v), the cooling systems 1, 2, 3, and/or 4 may include a terminal cooling element 120. In some cases, the terminal cooling element 120 may be configured to interface with insert 101 and be in fluid communication with the one or more lumens (or fluid pathways) of insert 101. Accordingly, in some cases, the terminal cooling element may be configured to extend the fluid pathways of insert 101. In some more cases, the terminal cooling element 120 may be integrally formed with insert 101 and the lumen(s) of insert 101 may extend within terminal cooling element 120. In some additional cases, terminal cooling element 120 may be configured to interface with track 115 of blade 110. More details of the terminal cooling element 120 are described below.

In accordance with some embodiments, the terminal cooling element 120 may be manufactured from one or more of the following materials: stainless steel, aluminum, titanium, polymer composites, or biocompatible alloys, or combinations thereof. In some instances, preferably, the terminal cooling element 120 may be manufactured from a metal or alloy thereof to enhance the thermal conductivity of the secondary cooling element 120. In some cases, terminal cooling element 120 may be substantially rigid. In accordance with some other cases, terminal cooling element 120 may be substantially flexible. In still some more embodiments, terminal cooling element 120 may comprise malleable sections, rigid sections, or a combination thereof to allow for both flexibility and structural stability.

Also, as previously described above for the various other elements of cooling systems 1, 2, 3, and/or 4 (i.e., blade 110, track 115, insert 101, etc.), the longitudinal extent (e.g., length) and transverse extent (e.g., width or diameter) of the terminal cooling element 120 may be customized, as desired. In some embodiments, the terminal cooling element 120 may have a length in the of about 20mm – 300mm (e.g., about 20 – 50 mm, about 50 – 150 mm, about 150 – 300mm, or any other subrange in range of about 20 – 300 mm). In some embodiments, the terminal cooling element 120 may have a width or diameter in the range of about 10 – 40 mm (e.g., about 10 – 20 mm, about 20 – 30 mm, about 30 – 40mm, or any other subrange in range of about 10 – 40 mm). In some embodiments, the secondary cooling element 120 may have a substantially uniform width, diameter, and/or depth along its entire length. In some additional embodiments, the terminal cooling element 120 may have a non-uniform (e.g., tapered, flared, ribbed, notched, constricted, bulged, or otherwise varying) width, diameter, and/or depth along its entire length. In some embodiments, the terminal cooling element 120 may have substantially different geometries than insert 101 (e.g., a greater or smaller length, width, and/or depth). As will be appreciated in light of this disclosure, it generally may be desirable to ensure that secondary cooling element 120 is sufficiently sized and of appropriate geometry to permit interfacing with track 115, blade 110 and/or insert 101.

In accordance with some embodiments, secondary cooling element 120 may have one more inlet(s) and one or more outlet(s) configured to interface and be in fluid communication with one or more inlet(s) and one or more outlet(s) of the insert 101. In some instances, for example, the terminal cooling element 120 may be configured to extend the closed fluid pathway in the insert 101.

In accordance with some embodiments, terminal cooling element 120 may include one or more lumen(s). Also, generally the longitudinal extent (e.g., length) and transverse extent (e.g., width or diameter) of the lumens of terminal cooling element 120 may be customized, as desired, and will depend on the overall dimensions of the terminal cooling element 120. In some embodiments, the lumen(s) may have a substantially uniform width, diameter, and/or depth along its entire length. In some additional embodiments, the lumen(s) may have a non-uniform (e.g., tapered, flared, ribbed, notched, constricted, bulged, or otherwise varying) width, diameter, and/or depth along its entire length. In some additional embodiments, the lumen(s) may terminate in a reservoir located within the terminal cooling element 120. As will be appreciated in light of this disclosure, it generally may be desirable to ensure that the lumen(s) are sufficiently sized and of appropriate geometry to permit fluid flow through the lumens within terminal cooling element 120.

In accordance with some embodiments, the shape of the lumen pathway (fluid pathway), or otherwise the path the fluid takes, may be customized, as desired. In some embodiments, the lumen(s) may be generally straight, curved, zig-zagged, or otherwise formed to provide a pathway from one internal volume portion of the terminal cooling element 120 to another internal volume portion of the terminal cooling element 120. As will be appreciated in light of this disclosure, it generally may be desirable to ensure the one or more internal fluid pathway(s) (i.e., the one or more lumen(s)) are sufficiently designed to permit an efficient transfer of thermal energy from a fluid traveling inside the lumens to the material forming the lumen(s) and/or terminal cooling element 120 and the blade 110 interfaced with the terminal cooling element 120, or vice versa.

In accordance with some embodiments, the one or more lumens may include a copper element extending along at least a partial extent of the one or more lumen(s), in whole or in part. The copper element may be positioned within the lumen, on an inside wall of the lumen, and/or on an outside wall of the lumen. The copper element may also be, in some cases, positioned adjacent to, proximate to, or distal from the lumen on insert 101 of cooling device(s) 1. Generally, the copper element may be positioned anywhere on and/or within terminal cooling element 120 to help aid in shaping and/or conforming terminal cooling element 120 to a specific shape and/or retaining terminal cooling element in a shape. The copper element may be a thin strand of copper wire configured to help with contouring the terminal cooling element 120 to the blade 110 and/or a patient body/cavity.

In accordance with some embodiments, terminal cooling element 120 may contain one or more recesses 121. In general, and as can be seen from FIGS. 2A(ii) – 2A(iii), the recess(es) 121 may generally be configured to enable the coupling, attaching, and/or interfacing between an insert 101 and the terminal cooling element 120. In other cases, the recess(es) 121 may allow for particular arrangement and/or placement of insert 101 when insert 101 is integrally formed with terminal cooling element 120. For example, in some cases, a recess positioned to receive at least a portion of the insert and align at least one inlet and outlet of the terminal cooling element 120 with a respective outlet and inlet of the insert 101. In some further cases, the terminal cooling element 120 may have a plurality of inlets and outlets aligning with a plurality of inlet and outlets of the insert 101. According to some embodiments, the recess(es) 121 may be formed centrally on and/or within a side of terminal cooling element 120 and/or biased towards one edge of terminal cooling element 120.

In accordance with some embodiments, the insert 101 may be integrally formed with or be configured to couple, attach, or otherwise interface with terminal cooling element 120 using adhesive, sealant, overmolding techniques, mechanical fasteners, and/or friction fit. Other suitable types of coupling mechanisms for coupling terminal cooling element 120 and insert 101 may depend on a given target application or end-use and will be apparent in light of this disclosure.

In accordance with some embodiments, terminal cooling element 120 may include one or more protrusion(s) 118. In general, the protrusion(s) may be configured to enable interfacing between the blade 110 and/or track 115 and the terminal cooling element 120. In some embodiments, the protrusion(s) may be configured to interface with one or more recess(es) on the blade 110 (i.e., either in the track 115 and/or on another portion of blade 110). For example, as can be seen in FIGS. 1B(i) – 1B(iii) and 2A(i) – 2A(v), terminal cooling element 120 may have protrusion 118 at a distal end, allowing terminal cooling element 120 to slot into a corresponding recess in a distal end of blade 110, thereby securely coupling the terminal cooling element 120 and the blade 110 together. Other suitable types, or combination of types, of methods to couple, interface, or otherwise securely fit the terminal cooling element 120 and the blade 110 together will depend on a given target application or end-use and will be apparent in light of this disclosure. For example, generally, terminal cooling element 120 may be configured to interface with blade 110. The interface between blade 110 and terminal cooling element 120 may use one or more of the following securing methods: snap-fit, slide-fit, friction-fit, and slot-fit. In some additional embodiments, adhesive, screws, tape, or combination thereof may be used in conjunction with the securing methods to achieve a secure interface between blade 110 and insert 101. Other suitable types, or combination of types, of securing methods as described herein will depend on a given target application or end-use and will be apparent in light of this disclosure.

In accordance with some embodiments, and as can be seen in at least FIGS. 1A(i) – 1A(v), the terminal cooling element 120 may be contoured to match the face of the blade 110. In such cases, this may produce a continuous smooth surface when the terminal cooling element 120 may be interfaced with blade 110 via track 115.

### Cooling Device(s) with Malleable Additions

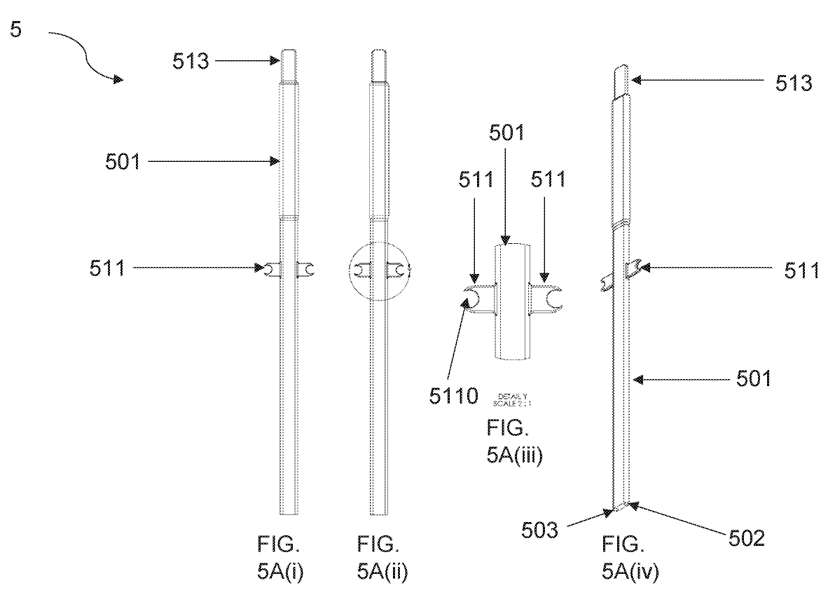

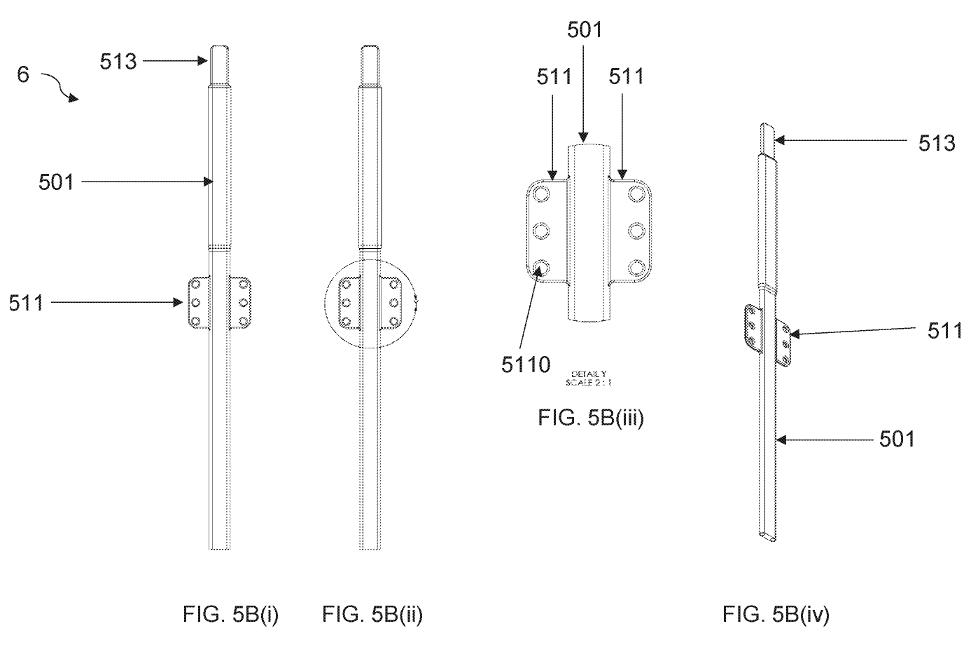

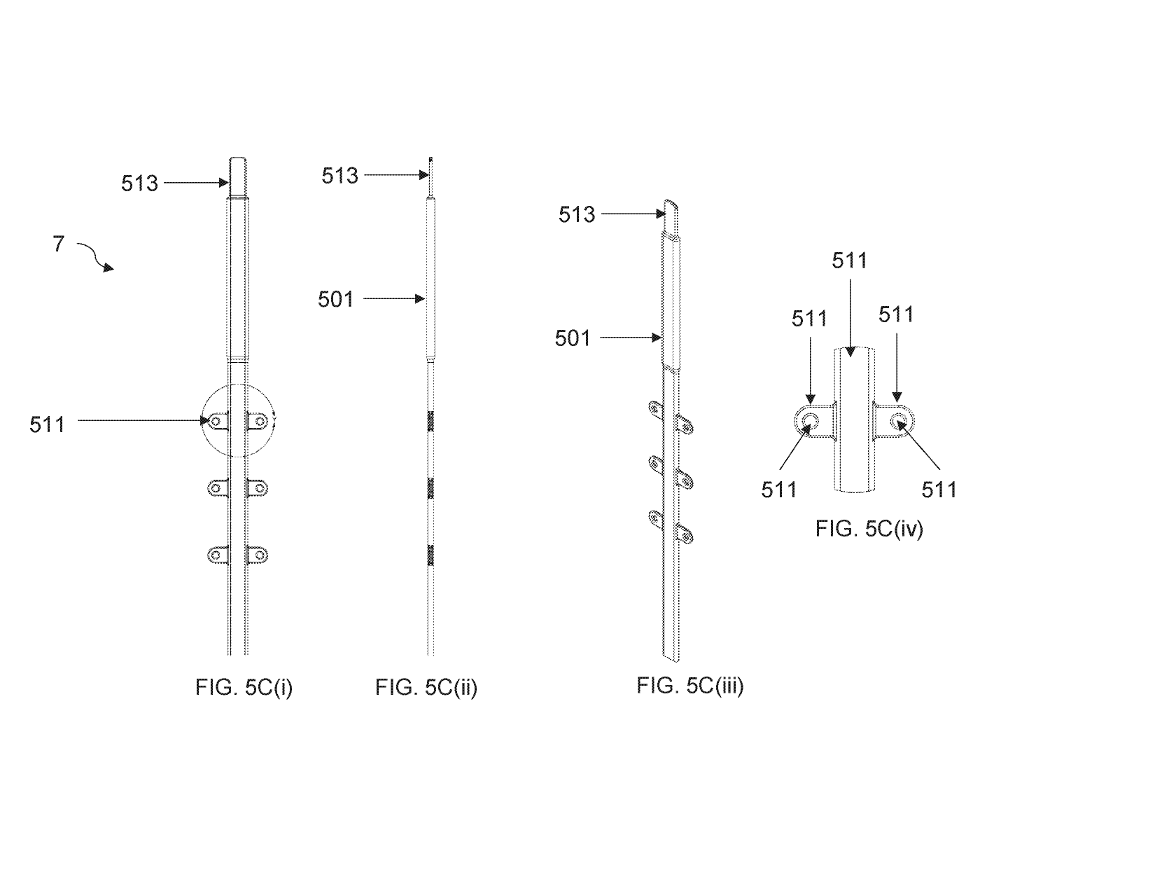

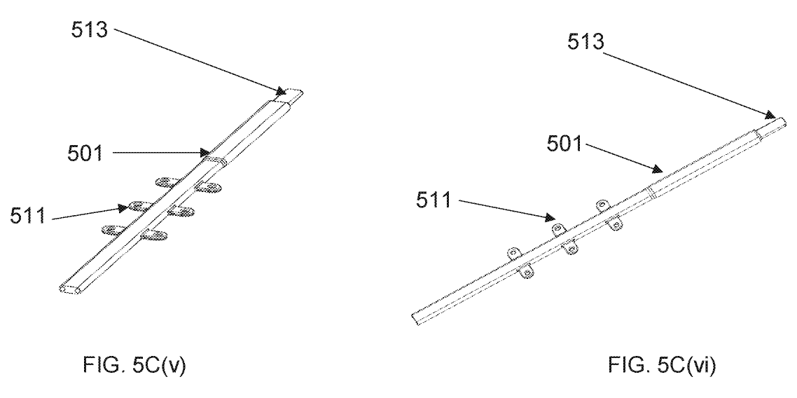

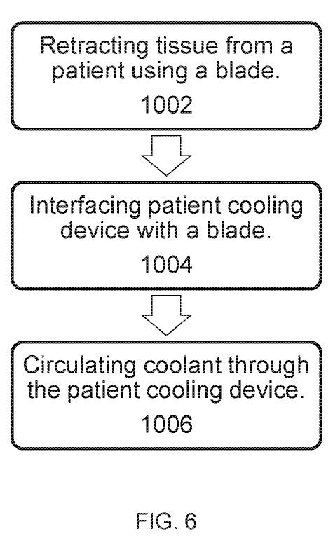

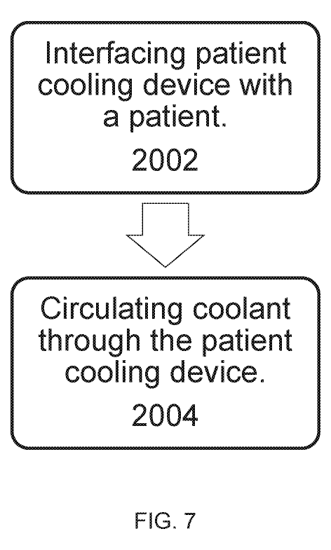

FIGS. 5A(i) – 5C(vi) show various exemplary embodiments of cooling device(s) disclosed herein. More specifically, FIGS. 5A(i) – 5A(iv) show an exemplary embodiment of cooling device 5, FIGS. 5B(i) – 5B(iv) show an exemplary embodiment of cooling system 6, and FIGS. 5C(i) – 5C(vi) show an exemplary embodiment of cooling system 7. It is to be understood that some features discussed with respect to some embodiments of cooling device 5, 6 and/or 7 may be common to other embodiments of cooling devices 5, 6 and/or 7. Accordingly, common reference numerals and/or descriptions are used herein to describe the various features of cooling devices 5, 6 and/or 7.

In accordance with some embodiments, cooling device(s) 5, 6, and/or 7 may include a body 501 having one or more lumens configured to interface with, or otherwise be in fluid communication with, one or more inlets 502 and/or one or more outlets 503. In accordance with some embodiments, the one or more lumens may extend within body 501 and form a continuous fluid pathway from one or more inlets 502 to one or more outlets 503 within body 501. In accordance with some embodiments, cooling device(s) may include one or more appendages 211 extending from the body 201. In some cases, the appendages 511 may include one or more eyelets (or holes) 5110. Additionally, in accordance with some embodiments, the cooling device(s) may include a protrusion 513 at least one end of the cooling device(s). Each of these elements is discussed in turn below.

In general, the cooling device(s) 5, 6, and/or 7 may include a body 501. In accordance with some embodiments, body 501 may be manufactured from one or more of the following materials: stainless steel, aluminum, titanium, polymer composites, or biocompatible alloys, or combinations thereof. In some cases, body 501may be substantially rigid. In accordance with some other cases, body 501may be substantially flexible. In still some more embodiments, body 501 may comprise malleable sections, rigid sections, or a combination thereof to allow for both flexibility and structural stability.

Also, the longitudinal extent (e.g., length) and transverse extent (e.g., width or diameter) of body 501 may be customized, as desired. In some embodiments, body 501 may have a length in the range of about 3mm – 50mm in (e.g., about 3 – 10 mm, about 10 – 25 mm, about 25 – 50 mm, or any other subrange in range of about 3 – 50 mm). In some embodiments, body 501 may have a width or diameter in the range of about 3 – 30 mm (e.g., about 3 – 8 mm, about 8 – 15 mm, about 15 – 30 mm, or any other subrange in range of about 3 – 30 mm). In some embodiments, body 501 may have a substantially uniform width, diameter, and/or depth along its entire length. In some additional embodiments, body 501 may have a non-uniform (e.g., tapered, flared, ribbed, notched, constricted, bulged, or otherwise varying) width, diameter, and/or depth along its entire length. As will be appreciated in light of this disclosure, it generally may be desirable to ensure that body 501 is sufficiently sized and of appropriate geometry to permit interfacing with a patient body/cavity.

Regarding body 501 of cooling device(s) 5, 6, and/or 7, the general shape thereof may be customized, as desired. In some embodiments, body 501 can be generally polygonal (e.g., triangular, rectangular, square, etc.) in shape. For example, in some cases, as can be seen from FIGS. 5A(i) – 5C(vi), body 501 may be of generally rectangular shape. Other suitable shapes, or combinations of shapes, of body 501 will depend on a given target application or end-use and will be apparent in light of this disclosure.

As explained above, body 501 may include one or more lumen(s). In accordance with some embodiments, the lumen(s) may be internal fluid pathways in fluid communication with one or more inlet(s) 502, one or more outlet(s) 503, and/or one or more additional lumen(s). Generally, the inlet(s) 502, the outlet(s) 503, and/or the one or more lumen(s) may be a part of a closed fluid loop with one or more additional cooling device(s) and/or systems. In accordance with some embodiments, the lumen(s) may be embedded, in whole or in part, within body 501. In some cases, the lumens may be circular, elliptical, star-shaped, oval, and/or rectangular in cross-sectional shape, in part or in whole. Other suitable types, or combinations of types, of cross-sectional shapes as described herein will depend on a given target application or end-use and will be apparent in light of this disclosure.

Also, generally the longitudinal extent (e.g., length) and transverse extent (e.g., width or diameter) of the lumens of body 501 may be customized, as desired, and will depend on the overall dimensions of the body 501. In some embodiments, the lumen(s) may have a substantially uniform width, diameter, and/or depth along its entire length. In some additional embodiments, the lumen(s) may have a non-uniform (e.g., tapered, flared, ribbed, notched, constricted, bulged, or otherwise varying) width, diameter, and/or depth along its entire length. In some additional embodiments, the lumen(s) may terminate in a reservoir located within the body 501. As will be appreciated in light of this disclosure, it generally may be desirable to ensure that the lumen(s) are sufficiently sized and of appropriate geometry to permit fluid flow through the lumens within the body 501.

In accordance with some embodiments, the shape of the lumen pathway, may be customized, as desired. In some embodiments, the lumen(s) may be generally straight, curved, zig-zagged, or otherwise formed to provide a pathway from one internal volume portion of the body 501 to another internal volume portion of the body 501. As will be appreciated in light of this disclosure, it generally may be desirable to ensure the one or more internal fluid pathway(s) (i.e., the one or more lumen(s)) are sufficiently designed to permit a transfer of thermal energy in an efficient manner.

In accordance with some embodiments, the one or more lumen(s) may be in fluid communication at a distal end of body 501 via a reservoir, a connecting tube, inlet(s), outlet(s), or combinations thereof. For example, in some cases, an inlet 502 may be in fluid communication with a lumen which extends at least partially within body 501. In some additional cases, the lumen(s) may be designed or configured to be in fluid communication with both inlet(s) 502 and outlet(s) 503 such that fluid may enter the inlet(s) 502, flow through the lumen within body 501, and then exit outlet(s) 503. In some additional cases, the outlet(s) 503 may be configured as inlet(s) 502 and the inlet(s) 502 may be configured as outlet(s) 503.

In accordance with some embodiments, the one or more lumens may include a copper element extending along at least a partial extent of the one or more lumen(s), in whole or in part. The copper element may be positioned within the lumen, on an inside wall of the lumen, and/or on an outside wall of the lumen. The copper element may also be, in some cases, positioned adjacent to, proximate to, or distal from the lumen on body 501 of cooling device(s) 5, 6, and/or 7. Generally, the copper element may be positioned anywhere on and/or within body 501 to help aid in shaping and/or conforming body 501 to a specific shape and/or retaining body 501 in a shape. The copper element may be a thin strand of copper wire configured to help with contouring the cooling device(s) 5, 6, and/or 7 to the anatomy of the patient body part where the cooling device(s) is being used.

As explained above, and as can be seen from FIGS. 5A(i) – 5C(vi), cooling device(s) 5, 6, and/or 7 may include one or more appendages 511 extending from the body 501. The appendages 511 may be formed as a constituent element of body 501, or may be manufactured separately and attached, coupled, or otherwise interfaced onto and/or with body 501. In accordance with some embodiments, body 501 may be manufactured from one or more of the following materials: stainless steel, aluminum, titanium, polymer composites, or biocompatible alloys, or combinations thereof. In some cases, body 501 may be substantially rigid. In accordance with some other cases, body 501 may be substantially flexible. In still some more embodiments, body 501 may include malleable sections, rigid sections, or a combination thereof to allow for both flexibility and structural stability. In some additional embodiments, the appendages 511 may be formed as the same constituent elements as body 501, a combination of constituent elements, or from different elements as body 501.

According to some embodiments, as can be seen from FIGS. 5A(i) – 5C(vi), the appendages 511 may be positioned along any part of the longitudinal or transverse extent of body 501. For example, as can be seen from FIGS. 5C(i) – 5C(vi), a pair of appendages 511 (e.g., a first appendage 511 with a second appendage 511 mirrored on the other side of body 501), may be positioned at a distal portion of body 501 and a second pair of appendages 511 may be positioned at a proximate portion of body 501. In some more cases, the appendages 511 may be positioned offset from one another along the longitudinal or latitudinal extent of body 501, such that one appendage 511 does not have a mirrored appendage across from it. Other suitable locations, or combination of locations, of appendages 511 on body 501 will depend on a given target application or end-use and will be apparent in light of this disclosure.

Also, the longitudinal extent (e.g., length) and transverse extent (e.g., width or diameter) of the appendages 511 may be customized, as desired. In some embodiments, appendages 511 may have a length in the range of about 1–10 mm (e.g., about 1–2, 2–4, 4–6, 6–10 mm, or any other subrange in the range of about 1–10 mm). In some embodiments, appendages 511 may have a width or diameter in the range of about 1–10 mm (e.g., about 1–2, 2–4, 4–6, 6–10 mm, or any other subrange in the range of about 1–10 mm). In some embodiments, appendages 511 may have a substantially uniform width, diameter, and/or depth along its entire length. In some additional embodiments, appendages 511 may have a non-uniform (e.g., tapered, flared, ribbed, notched, constricted, bulged, or otherwise varying) width, diameter, and/or depth along its entire length. As will be appreciated in light of this disclosure, it generally may be desirable to ensure that appendages 511 are sufficiently sized and of appropriate geometry to permit interfacing with a patient body/cavity.

Regarding appendages 511 of body 501, the general shape thereof may be customized, as desired. In some embodiments, appendages 511 can be generally polygonal (e.g., triangular, rectangular, square, etc.) in shape, or generally circular, elliptical, semi-circular, or semi-elliptical in shape, or combinations thereof. Other suitable shapes, or combinations of shapes, of appendages 511 will depend on a given target application or end-use and will be apparent in light of this disclosure.

According to some embodiments, and as can be seen in FIGS. 5B(i) – 5C(vi), the appendages 511 may include one or more eyelets (or holes) 5110. For instance, as can be seen in FIGS. 5B(i) – 5B(iv), appendages 511 have a plurality of holes 5110 within one appendage 511. In other embodiments, for instance as can be seen from FIGS. 5C(i) – 5C(vi), appendages 511 have a single hole 5110 within appendage 511. The one or more eyelets (holes) 5110 may be positioned anywhere on the transverse or longitudinal extent of the appendages 511. As a non-limiting example, as seen in FIG. 5C(i), the eyelet 5110 may be positioned at a distal portion of the appendage from the body 501. In some additional cases, the eyelet(s) 5510 may generally be circular, elliptical, semi-circular, or semi-elliptical in cross-sectional shape. For example, as can be seen from FIG. 5C(i), the eyelet 212 is circular. However, in some additional cases as can be seen in FIGS. 5A(i) – 5A(iv), the eyelet(s) 5510 may be positioned at an edge of appendages 511 such that the eyelet(s) 5110 have a substantially C-shape. In some cases, the one or more eyelet(s) 5110 may have a uniform shape or have a combination of cross-sectional shapes, in whole or in part. For example, in some instances the one or more eyelet(s) 5110 may have a uniform diameter throughout the extent of the one or more eyelet(s) 5110. In some other instances, the diameter of the one or more eyelet(s) 5110 may change (e.g., increase or decrease) along the depth of the one or more eyelet(s) 5110. As may be generally appreciated, the eyelet(s) 5110 may be configured to allow sutures and/or suture needles to pass therethrough and/or attach body 501 to a patient via sutures and/or other medical fasteners. Other suitable types, or combination of types, of cross-sectional shapes, as described herein, will depend on a given target application or end-use and will be apparent in light of this disclosure.

According to some embodiments, as can be seen in FIGS. 5A(i) – 5C(vi), the body 501 may include a protrusion 513 at least one end of the body 501. In some cases, the protrusion 513 may be appropriately sized and/or configured to interface with one or more portions of a human body before, after, and/or during a surgical procedure. In some cases, as a specific, non-limiting example, the protrusion 513 may be configured to fit, or otherwise be positioned, under and/or around the superior or inferior lamina after a laminectomy.

In accordance with some embodiments, protrusion 513 may be manufactured from one or more of the following materials: stainless steel, aluminum, titanium, polymer composites, or biocompatible alloys, or combinations thereof. In some cases, body 501may be substantially rigid. In accordance with some other cases, body 501may be substantially flexible. In still some more embodiments, body 501may comprise malleable sections, rigid sections, or a combination thereof to allow for both flexibility and structural stability. In some cases, the protrusion 513 may extend from the body 501. The protrusion 513 may be formed as a constituent element of body 501, or may be manufactured separately and attached, coupled, or otherwise interfaced onto and/or with body 501. In additional cases, the protrusion may be manufactured, or otherwise is, overmolded from body 501. In some additional cases, protrusion 513 may be attached, interfaced with, or otherwise coupled to body 501 using adhesive, sealant, and/or other suitable coupling techniques.

Also, the longitudinal extent (e.g., length) and transverse extent (e.g., width or diameter) of protrusion 513 may be customized, as desired. In some embodiments, protrusion 513 may have a length in the range of about 3mm – 50mm in (e.g., about 3 – 10 mm, about 10 – 25 mm, about 25 – 50 mm, or any other subrange in range of about 3 – 50 mm). In some embodiments, protrusion 513 may have a width or diameter in the range of about 3 – 30 mm (e.g., about 3 – 8 mm, about 8 – 15 mm, about 15 – 30 mm, or any other subrange in range of about 3 – 30 mm). In some embodiments, protrusion 513 may have a substantially uniform width, diameter, and/or depth along its entire length. In some additional embodiments, protrusion 513 may have a non-uniform (e.g., tapered, flared, ribbed, notched, constricted, bulged, or otherwise varying) width, diameter, and/or depth along its entire length. In some cases, the protrusion 513 may be flat (i.e., the longitudinal and/or transverse length of the protrusion 513 is noticeably greater than the depth of the protrusion 513). As will be appreciated in light of this disclosure, it generally may be desirable to ensure that protrusion 513 is sufficiently sized and of appropriate geometry to permit interfacing with a patient body/cavity.

Regarding the protrusion 513 of cooling device(s) 5, 6, and/or 7, the general shape thereof may be customized, as desired. In some embodiments, protrusion 513 can be generally polygonal (e.g., triangular, rectangular, square, etc.) in shape. For example, in some cases, as can be seen from FIGS. 5A(i) – 5C(vi), protrusion 513 may be of generally rectangular shape. Other suitable shapes, or combinations of shapes, of protrusion 513 will depend on a given target application or end-use and will be apparent in light of this disclosure.

According to some embodiments, the protrusion 513 may include one or more lumens in fluid communication with the one or more lumens of body 201. In some other embodiments, the protrusion 513 may be devoid of lumens and, as such, may not directly contribute to the thermal therapy.

### Method 1000

In accordance with some embodiments, the cooling device(s) and systems disclosed herein may be configured to perform one or more functions or methods. Various uses for the cooling device(s) and systems may be devised by one of ordinary skilled in the art and in light of this disclosure.

In some embodiments, the method 1000 may include, as in block 1002, retracting tissue away from a target site using one or more exemplary cooling systems 1, 2, 3, and/or 4, in part or in whole. More specifically, the tissue may be retracted using blade 110. Additionally, cooling systems 1, 2, 3, and/or 4 may be configured to hold itself in place or interface with another element (such as a mounting system) to hold cooling systems 1, 2, 3, and/or 4 in place during a surgical procedure.

Generally, the method 1000 may also include interfacing patient cooling device(s) 1a, 2a, 3a, and/or 4a with blade 110. More specifically, insert 101 and/or terminal cooling element 120 with blade 110. The insert 101 may or may not have terminal cooling element 120 interfaced with insert 101 at the time of interfacing insert 101 with blade 110. In some cases, both the insert 101 and the terminal cooling element 120 may be interfaced with blade 110, using or not using track 115. As discussed above, blade 110 may include one or more elements (such as track 115 and or recesses) configured as interfacing locations for insert 101 and/or terminal cooling element 120. The method 1000 may also, as seen in block 1006, include circulating coolant through the one or more lumens of insert 101 and/or terminal cooling element 120, thereby applying cooling to blade 110 and/or the retracted tissue and/or tissue adjacent to blade 110.

Regarding the surgical procedure, cooling systems 1, 2, 3, and/or 4 may generally be used in any surgical procedure which may require a retracting blade. For example, some surgical procedures may include or otherwise be associated with general spine surgery. In some cases, as a non-limiting example, in lateral lumbar access spine surgery that includes a trans-psoas approach, one or more cooling systems 1, 2, 3, and/or 4 may be inserted and dissect the psoas muscle. Compression of the nerves in the region may cause patients to experience pain and/or damage, which may be reduced by the cooling effect of the fluid flowing into and/or through the one or more cooling systems 1, 2, 3, and/or 4. In some additional cases, as another non-limiting example, during surgeries which require anterior cervical spine access and thus the retraction of the esophagus and trachea, the hoarseness, dysphasia, and/or pain may reduced by the cooling effect of the fluid flowing into and/or through the one or more cooling systems 1, 2, 3, and/or 4 used in the retraction of the esophagus and trachea. Other surgical procedures where it may be beneficial to apply local hypothermia at a site of retraction using a retractor blade (or otherwise the cooling systems 1, 2, 3, and/or 4, as described herein) may be apparent in light of this disclosure. In general, the cooling device 1 may reduce mechanisms of injury caused by retraction systems, including inflammation, cell damage and death, and ischema.

In accordance with some embodiments, the method 1000 may include removing insert 101 from blade 110. In some cases, insert 101 may be removed by inserting, through an opening located at the distal end of blade 110, a tool to disengage insert 101 from track 115 and therefore blade 110. It is to be understood that the cooling systems 1, 2, 3, and/or 4 disclosed herein may accommodate any suitable type of fluid and/or coolant. For example, in some embodiments, the fluid and/or coolant may be a liquid or a gas. For example, in some instances, the fluid and/or coolant used may be one or more of the following fluids: saline solution, water, liquid nitrogen, ethyl alcohol, or combinations thereof.

In accordance with some embodiments, the method 1000 may include attaching, interfacing, or otherwise coupling a terminal cooling element 120 to insert 101. In some cases, for example, insert 101 may be configured to slot into, or otherwise couple with, terminal cooling element 120 thereby extending the lumens of the insert by having them be in fluid communication with the lumens of the terminal cooling element 120. In such cases, the terminal cooling element 120 may further be attached, interfaced, or otherwise coupled with track 115 of blade 110.

### METHOD 2000

In accordance with some embodiments, cooling device 2 may be configured to perform one or more functions or methods. Various uses for cooling device 2 may be devised by one of ordinary skilled in the art and in light of this disclosure.

In some embodiments, the method 2000 may (optionally) include placing the cooling device(s) 5, 6, and/or 7 on a portion of a patient’s anatomy. In some cases, the method 2000 may include attaching, securing, or otherwise coupling the cooling device(s) 5, 6, and/or 7 to the patient. In some more cases, the method 2000 may include bending, maneuvering, or otherwise moving cooling device(s) 5, 6, and/or 7 to conform with the contours of the patient’s anatomy. In some additional cases, the method 2000 may include maneuvering the protrusion to a location of the patient’s body. The method 2000, in some more cases, may include circulating coolant through cooling device(s) 5, 6, and/or 7 to provide thermal therapy to an area of the patient. The method 2000 may (optionally), include reducing the size of the appendages and/or the protrusion before coupling the cooling device(s) 5, 6, and/or 7 to the patient. The method 2000 may (optionally) include closing an incision containing the cooling device(s) 5, 6, and/or 7 to permit thermal therapy in a post-operative environment.

As stated above, in accordance with some embodiments the method 2000 may (optionally) include placing the cooling device(s) 5, 6, and/or 7 on a portion of a patient’s anatomy. In some cases, as a non-limiting example, the cooling device(s) may be placed on an exposed dura mater of a patient after a laminectomy. Other suitable locations for cooling device(s) before, during, and/or after a surgical procedure will depend on the given target application or end-use and will be apparent in light of this disclosure.

In accordance with some embodiments, the method 2000 may include attaching, securing, or otherwise coupling cooling device(s) 5, 6, and/or 7 to the patient. In some cases, a suture needle and attached suture may be passed through one or more of the eyelets and into a region of the patient in order to secure the cooling device(s) to the patient. In general, the eyelet(s) 5110 of cooling device(s) 5, 6, and/or 7 may enable a user to suture the cooling device(s) onto a patient.

In accordance with some embodiments, the method 2000 may include bending, maneuvering, or otherwise moving cooling device(s) 5, 6, and/or 7 to conform with the contours of the patient’s anatomy. In some cases, a user may bend the cooling device(s) 5, 6, and/or 7 to conform with a curve and/or shape of the location the user wishes to place the cooling device on the patient. In doing so, in some instances, the copper element may be configured to provide adequate rigidity and allow the cooling device(s) 5, 6, and/or 7 to maintain the substantially same shape.

In accordance with some embodiments, the method 2000 may include maneuvering the protrusion 513 to a location of the patient’s body. In some cases, for example, the protrusion may be tucked or placed under the inferior or superior lamina of a patient during a laminectomy. Other suitable locations for the protrusion before, during, and/or after a surgical procedure will depend on the given target application or end-use and will be apparent in light of this disclosure.

In accordance with some embodiments, the method 2000 may (optionally) include circulating coolant through cooling device(s) 5, 6, and/or 7 to provide thermal therapy to an area of the patient. The cooling device(s) may be configured to interface and be in fluid communication with a cooling system to provide cooling fluid to the cooling device(s). The cooling system may pump fluid into the inlet(s) of the cooling device(s), which subsequently follows the path of the lumen(s) through the cooling device(s) and exits the outlet(s).

In accordance with some embodiments, the method 2000 may (optionally) include reducing the size of the appendages and/or the protrusion before coupling the cooling device(s) 5, 6, and/or 7 to the patient. For example, in some cases, the appendages may be configured to permit a user to re-size the appendage to better conform with the anatomy of the patient. In such instances, the appendages may be cut or otherwise reduced to ensure an adequate fit with the patient.

In accordance with some embodiments, the method 2000 may (optionally) include closing an incision containing the cooling device(s) 5, 6, and/or 7 to permit thermal therapy in a post-operative environment. For example, in some cases, a user may decide to leave the cooling device(s) on the patient and close any incision created to allow the insertion of the cooling device into the patient, thereby at least partially enclosing the cooling device(s) within the patient. In such cases, this may allow post-operative cooling while maintaining a lower degree of risk to the area of interest due to infection.

The foregoing description of example embodiments has been presented for the purposes of illustration and description. It is not intended to be exhaustive or to limit the present disclosure to the precise forms disclosed. Many modifications and variations are possible in light of this disclosure. It is intended that the scope of the present disclosure is not limited by this detailed description. Future-filed applications claiming priority to this application may claim the disclosed subject matter in a different manner and generally may include any set of one or more limitations as variously disclosed or otherwise demonstrated herein.

What is claimed is:

1. A cooling system for applying thermal therapy to a tissue of a patient, the cooling system comprising:
a retractor blade comprising an interfacing location extending along at least a portion of a length of the retractor blade; and
a cooling attachment configured to couple with the interfacing location, the attachment comprising:
an inlet configured to interface and be in fluid communication with a fluid supply;
a lumen in fluid communication with the inlet and extending at least partially within the cooling attachment; and
an outlet in fluid communication with the lumen and configured to interface and be in fluid communication with a fluid return.

2. The cooling system of claim 1, wherein the interfacing location is on a tissue contacting side of the retractor blade.

3. The cooling system of claim 1, wherein the cooling attachment is engaged with the interfacing location by an interfacing technique selected from a group consisting of: snap-fit, slot-fit, adhesives, fasteners, or combinations thereof.

4. The cooling system of claim 3, wherein the cooling attachment is snap-fit within the interfacing location.

5. The cooling system of claim 1, wherein the cooling attachment further comprises:
an insert configured to couple with the blade; and
a terminal cooling element configured to:
interface and be in fluid communication with an insert; and
couple to the interfacing location.

6. The cooling system of claim 5, wherein the insert is configured to carry fluid within the insert and to the terminal cooling element.

7. The cooling system of claim 5, wherein the terminal cooling element comprises a cavity configured to receive the insert and enable fluid communication between a lumen of the insert and a lumen of the terminal cooling element.

8. The cooling system of claim 5, wherein the terminal cooling element comprises metal.

9. The cooling system of claim 1, wherein a length of the interfacing location extends along less than half of a length of the blade.

10. The cooling system of claim 1, wherein a length of the interfacing location extends along half of a length of the blade.

11. The cooling system of claim 1, wherein a length of the interfacing location extends along a substantially full length of a face of the blade.

12. A patient cooling device configured to interface with a blade, comprising:
a tubing element; and
a terminal cooling element configured to be in fluid communication with the tubing element.

13. The patient cooling device of claim 12, wherein the tubing element is in fluid communication with an external fluid cooling system.

14. The patient cooling device of claim 12, wherein the terminal cooling element is configured to interface with an interfacing location of the blade.

15. The patient cooling device of claim 12, wherein the terminal cooling element is curved to match a curvature of the blade.

16. The patient cooling device of claim 12, wherein the patient cooling device is configured to interface on a tissue contacting side of the blade.
